(19) United States
(12) Patent Application Publication
(10) Pub. No.: US 2012/0156147 A1
(43) Pub. Date: Jun. 21, 2012

(54) COSMETIC COMPOSITION; SKIN TREATMENT KIT; METHOD FOR TREATING OILY OR MIXED SKIN OR ACNED SKIN

(75) Inventors: Ana Paula Pedroso de Oliveira, Sao Paulo SP (BR); Elaine Cristina de Oliveira, Sao Paulo SP (BR); Juliana Flor, Sao Paulo SP (BR); Livia Del Bosco, Sao Paulo SP (BR); Tiago Costa Beber, Sao Paulo SP (BR)

(73) Assignee: NATURA COSMETICOS S.A., Itapecerica da Serra - SP (BR)

(21) Appl. No.: 13/381,473

(22) PCT Filed: Jun. 29, 2010
(86) PCT No.: PCT/BR10/00205

§ 371 Date: Mar. 7, 2012

(30) Foreign Application Priority Data
Jun. 29, 2009 (FR) 0954417

(51) Int. Cl. A61K 8/49; A61Q 19/00; A61Q 19/08; A61Q 17/04

(52) U.S. Cl. 424/59

(57) ABSTRACT

The present invention refers to a cosmetic composition, preferably an oil-in-water emulsion (o/w) or aqueous gel, for the treatment of oily, mixed, or acned skin, comprising:—at least an anti-ageing sign active preferably corresponding to about 0.05% to about 10% of the total mass selected from the group consisting solubilized hydrophilic actives and lipophilic actives;—at least a sun protection system selected from the group consisting of hydrophilic sun-screens promoting photostable UVB and UVA protection, which preferably promotes a protection corresponding to a SPF of up to 30;—at least a oleosity control agent preferably comprising Zinc PCA; and—cosmetically acceptable alcohol-free adjuvants or carriers. The present invention is further directed to a skin treatmento kit preferably comprising a cosmetic composition as defined above for diurnal use and a complementary cosmetic composition, preferably a serum, for nighttime use. The present invention additionally refers to a skin treatment method comprising applying the cosmetic composition and complementary cosmetic composition.

### SUMMARY OF THE INVENTION

The present invention refers to cosmetic formulations, preferably an oil-in-water emulsion (o/w) or aqueous gel, for the treatment of oily, mixed or acned skin.

Further the invention refers to a skin treatment kit preferably comprising the above-mentioned cosmetic composition in a form of oil-in-water emulsion (o/w) or an aqueous gel for diurnal use and a complementary composition, which is an oil-in-water emulsion (o/w), preferably a serum, for night-time use.

Finally, the present invention refers to a skin treatment method comprising applying said cosmetic composition and complementary cosmetic composition.

### BACKGROUND OF THE INVENTION

Oily skin is characterized by excessive glow, uneven texture, dilated pores and increased thickness of the superficial layer, epidermis. This occurs because of excessive sebum production and also because of an increase in skin keratinization process. Such characteristics may have a negative aesthetic impact on a individual, leading to negative self-perception and impacting on social interactions (SEGOT-CHICQ et al., 2007), Furthermore, makeup on a oily skin lasts lesser than on normal and dry skins.

Sebum is an oily substance produced by sebaceous glands that are present on a major area of body surface, excepting for hand palm and feet sole, being it is found in a greater concentration on the scalp region, forehead and central face. The area of the body having the highest number of sebaceous glands is the face, where there can be found 400 to 900 glands per square centimeter (PUGLIESE, 2006). The so called T-zone of oleosity comprises forehead, nose and chin (ABRAMOVITS et al., 2000).

Sebaceous glands vary in size in accordance with the area and have a form similar to a pouch and on the superior portion a follicular channel. An aperture of this channel on skin surface is called pore. It is through this pore that hair comes out from the skin and that secretion of substances, such as sebum and sweat, occurs.

Sebum production is essential for keeping a healthy skin, once it is involved in the formation of hydrolipid layer that retains hydration and protects from external aggressions. Nevertheless, when in excess, sebum is harmful leaving the skin with a glow, greasy appearance and some times it causes acne formation due to obstruction, irritation and inflammation of pores.

Sebum production varies from person to person and depends on sex and age. Level of sebum production in the childhood and male and female prepuberty is very low. During adolescence, women have a higher level of sebum production than men, but sebum production rate is higher in men than in women due to the amount of testosterone hormone. Estrogen hormone in women has the function of suppressing sebum production; nevertheless high levels of androgen may inhibit estrogen. Such changes seem to be related to menstrual cycle and hormone replacement (PUGLIESE, 2006). In both men and women sebum production can be stimulated by physical or emotional factors which are altered by hormones. Sebum production slightly decreases as men age, while in women said production substantially decreases after 60 years of age.

Besides age and sex factors, sebum production is also influenced by stress (ZOUBOULIS, 2004).

Oily surface tends to form comedones (black spots, also called blackheads) dilated pores, excessive glow and sticky feel are characteristics which aid in identifying an oily skin.

Comedogenicity is the potential of a cosmetic/pharmaceutical product to cause comedones or pimples to be formed on the skin. Therefore, formulations defined as “non-comedogenic” exhibit less potential to cause formation of comedones or pimples on the skin.

Nowadays many commercially available cosmetic products are in the oil-free form, thereby making them more attractive for persons having an excessive production of sebum.

By labeling a product as oil-free it is not meant that it is non-comedogenic, since many raw materials other than oils are known to cause formation of blackheads or pimples on the skin. In order to evaluate non-comedogenic potential of a formulation, clinical tests with appropriate protocols must be carried out so as to indicate whether a product is suitable for oily skin consumers.

In addition to excessive sebum production, another extremely relevant factor relative to skin treatment is its ageing. Over time the skin is subjected to changes and slowdowns of several physiological processes. Not only do such changes happen on the epidermal layer (more superficial skin layer) but they also have an effect on the deeper layer of cutaneous skin.

Nowadays, lifestyle adopted by younger people, including smoking, stress, excessive sun exposure and pollution, might accelerate cutaneous ageing. From the age of thirty, there is a slowdown of some physiological process, such as, for example: cellular renovation, skin defense systems against free radicals as well as production of sustaining fibers such as collagen and elastin. Alteration in the production and regulation of said fibers has deleterious effects in both quality and amount of these fibers.

Many patent documents teach cosmetic compositions for protection and/or skin treatment. Nevertheless, none of them suggests an oil-in-water emulsion (o/w) or an aqueous gel for diurnal use, in addition to an oil-in-water emulsion (o/w), preferably a serum, for nighttime use in accordance with the present invention. Kit and skin treatment method also herein disclosed are not anticipated by the state of the art.

For example, U.S. Pat. No. 6,627,180 refers to a photoprotective topical cosmetic/dermatologic composition intended to be used for skin and/or hair photoprotection, comprising a synergistic combination of between: at least a photoprotective insoluble particulate organic material; at least an amino-substituted hydroxybenzophenone; and a physiologically acceptable carrier.

U.S. Pat. No. 6,699,461 refers to a photoprotective topical cosmetic/dermatologic composition intended for skin and/or hair photoprotection, comprising an effective amount of at least a photoprotective component of dibenzoylmethane; at least a filter stabilizer of hydroxybenzophenone; and a physiologically acceptable carrier.

U.S. Pat. No. 6,872,401 refers to topical cosmetic and/or dermatological compositions comprising, in a fatty base carrier, a tetrahydrocurcumin component or derivatives thereof; a stabilizer agent; and at least a solubilizing oil comprising at least a structural amide unit.

U.S. Pat. No. 6,994,844 refers to a topical photoprotective, photostable composition comprising: at least a dibenzoylmethane photoprotecting agent; and an effective amount of a dibenzoylmethane-4,4-diarylbutadiene stabilizer. It is clearly suggested therein that said composition is substantially free of any cinnamate.

U.S. Pat. No. 7,166,274 discloses a photoprotective cosmetic/dermatological composition for skin and/or hair protection, comprising: particles of at least an insoluble inorganic primary photoprotective agent; at least a secondary photoprotective agent; and an physiologically acceptable carrier.

U.S. Pat. No. 7,132,097 refers to a photoprotective composition comprising at least an active UV-B ingredient, optionally an active UV-A ingredient and 2-phenylethyl benzoate in a amount sufficient for increasing sunscreen protection factor of the final composition.

International Patent Application Publication WO 2007/135197 refers to a benzenophenone-4(2-hydroxy-4-methoxybenzophenone-5-sulfonic acid) composition for quenching fluorescence of disodium phenyl dibenzimidazole tetrasulfonate or salts thereof. Further it is explicitly mentioned that a synergistic combination between benzophenone-4 and disodium phenyl dibenzimidazole tetrasulfonate promotes an increase in UV radiation-absorbing properties of the latter component dispersed in cosmetic/dermatological compositions.

### DESCRIPTION OF THE DRAWINGS

FIG. 1 shows the results of a study carried out on ninety-five Brazilian women with ages ranging from 20 to 70 years, having different types of skin, users of cosmetic products, which demonstrated that skin elasticity decreases with ageing (NAKAMURA et al., 2005).

FIG. 2 depicts an increase in the production of tropoelastin (precursor of elastin) in a culture of fibroblasts treated with Elastinol +R (FROP's fraction) (ROBERT et al., 2004).

FIG. 3 depicts an increase in the dermis of Elastinol +R-treated collagen fiber density (FODIL-BOURAHLA et al., 2003).

FIGS. 4 and 5 depict an increase in the number of epidermis cells treated with Elastinol +R (Reports Nos. 2, 4 and 8).

FIG. 6 shows a comparison of increase in thickness of epidermis treated with Elastinol +R and Retinol (Report Nos. 37 and 39).

### DESCRIPTION OF THE INVENTION

The present invention refers to a cosmetic composition in the form of an oil-in-water emulsion (o/w) or aqueous gel, comprising:
at least an anti-ageing sign active preferably in an amount of about 0.05% to about 10.0% of total mass and selected from the group consisting of hydrophilic actives and solubilized lipophilic actives;
at least a sun protection system selected from the group consisting of hydrophilic sunscreens promoting photostable UV-B protection and UV-A protection, which preferably promotes protection corresponding to a SPF of up to 30;
at least an oil control agent, preferably comprising Zinc PCA; and
cosmetically acceptable alcohol-free adjuvants or carriers.

Preferably, said cosmetic composition additionally comprises at least an acne treatment agent comprising antiinflammatory actives and/or antiseptics and/or keratolytics from different origins.

Particularly, said composition is indicated for daylight use.

In addition, the present invention also refers to a skin treatment kit comprising a cosmetic composition as defined above, and, also a complementary composition, it preferably being in the form of an oil-in-water emulsion (o/w), and more preferably in a serum form, comprising:
at least an anti-ageing sign active in an amount of about 0.05% to about 10.0% of total mass and selected from the group consisting of solubilized hydrophilic actives and lipophilic actives;
salicylic acid is preferably present in an amount of about 0.1 to about 2.0% of total mass of the emulsion;
at least an oil control agent preferably comprising Zinc PCA; and
cosmetically acceptable adjuvants or carriers.

Preferably said complementary cosmetic composition is for nighttime use.

The present invention also refers to a method of treating oily, mixed skin or acned skin, comprising applying a cosmetic composition in the form of an oil-in-water emulsion (o/w) or an aqueous gel as defined above, during diurnal period. Preferably, said method further comprises applying a complementary cosmetic composition in the form of an oil-in-water emulsion (o/w) as defined above for nighttime use.

### DETAILED DESCRIPTION OF THE INVENTION

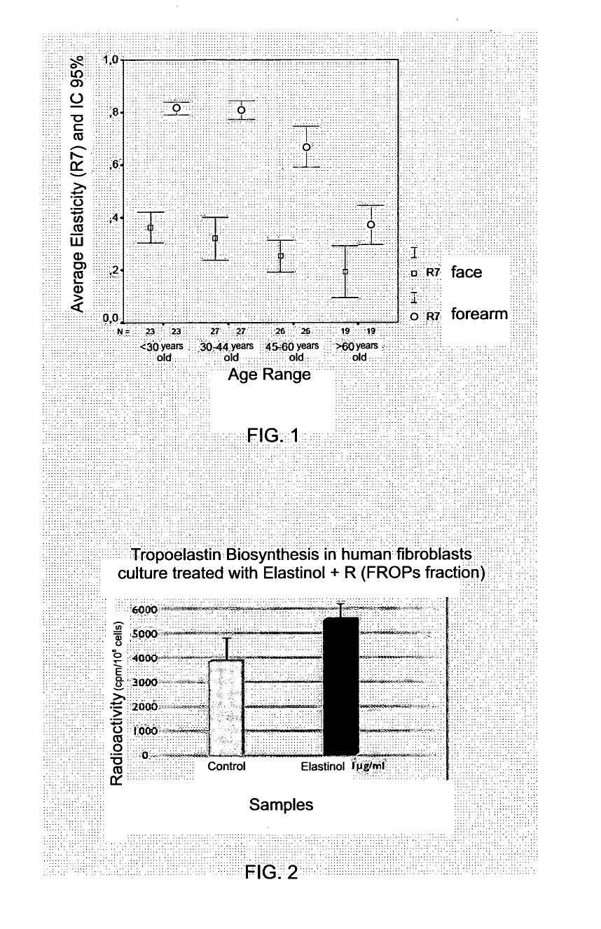

A study carried out on ninety-five Brazilian women with ages ranging from 20 to 70 years, having different types of skin, users of cosmetic products, showed that skin elasticity decreases with ageing (FIG. 1).

By this way, independent of skin types, strategies for treating ageing signs, such as elasticity lost and flaccidity must be considered in a cosmetic product, including oily skin.

Since there is a need for a product exhibiting effective sunscreen protection and anti-ageing system (anti-ageing signs) for oily skin persons, a cosmetic composition particularly in the form of an oil-in-water emulsion (o/w) or an aqueous gel, specially formulated for treating this type of skin, was developed. Said cosmetic composition of the present invention presents an extremely light, non-greasy, highly absorbed texture, and an immediate dry feel.

Said composition has a minimum Sunscreen Protection Factor (SPF) of 15 that protects skin from the action of UVB rays, in addition to highly protecting against UVA rays (90% protection by in vitro method—Australian methodology) that prevents premature ageing, and it keeps a photostable protection for 8 hours. It further comprises hydrosoluble sunscreens, thus rendering an ideal product for oily skin.

In addition to sunscreen protection, said composition comprises specific actives for hydration, oleosity and ageing signs treatment, such as:
at least a hydrating agent that hydrates the skin with leaving it oily;
at least an oleosity control agent for controlling excessive oleosity with continuous use;
at least one agent for treating acne, selected from the group of anti-inflammatory and/or antiseptic and/or keratolytic actives;
at least an anti-ageing sign active that promotes increase in collagen synthesis, regulates elastin production, promotes cellular renovation and redensifies the skin;
at least a film-forming agent that forms a micronet on the skin surface, promoting an immediate tensor effect.

Formulations of the present invention are also non-comedogenic, oil-free, stable and can optionally comprise fragrance and/or dyes.

Preferably, emulsions and gels in accordance with the present invention comprise at least an anti-ageing sign active selected from solubilized hydrophilic actives and lipophilic actives. Such anti-ageing sign actives are present in emulsions and gels of the present invention at a concentration ranging from about 0.05% to about 10% by mass of the total mass of the composition. Preferably, said anti-ageing sign active is a mixture of oligo- and polysaccharides rich in fucose and ramnose, herein designated as Elastinol +R, and is present in a concentration of about 5% of the total mass of the composition.

The formulation of the present invention further contemplates a sun protection system comprising only hydrophilic sunscreens which enhance protection against UVB rays, providing a Sunscreen Protection Factor (SPF) of up to 30, and against UVA rays. Additionally, said system is photostable and has the ability to keep sun protection for up 8 hours.

Preferably, hydrophilic sunscreens for protection against UVA and UVB rays are benzophonone-4, phenylbenzimizadole sulfonic acid and disodium phenyl dibenzimidazole tetrasulfonate.

Sun protection system is present in the emulsion and gels of the present invention in a concentration ranging from approximately 10% to 23%, preferably about 12.8% of total mass of the formulation.

Oleosity control agent is preferably Zinc PCA, which is present in the formulation in a concentration ranging approximately from 0.1% to 2.0% by mass, preferably about 0.70% of total mass of the composition.

As a hydrating agent, glycerin is preferably used and it is present in the emulsion and gels of the present invention in a concentration ranging from about 1% to 10%, preferably about 4% of total mass of the formulation.

The preferably used film-forming agent of the present invention is xanthan gum that confers a tensor effect on the skin. Said agent is present in the formulation in a concentration ranging from about 0.1% to 1.5%, preferably about 1% of total mass of the formulation. Other film-forming tensor effect agents, such as soy extract, rice extract, etc., can be used. Tensor effect is provided by ingredients that form a film on the skin surface.

Also included in said emulsions and gels are adjuvants selected from preservatives, consistency agents, emulsifiers, sensorial modifiers, and pH-correcting agents.

Preservatives present in said formulations are preferably phenoxyethanol and methylisothiazolinone, and together they are present in a concentration ranging from 0.4% to 1.0%, preferably about 0.49% by mass based on the total mass of the composition.

Sensorial modifiers present are selected from esters and silicones. Said sensorial modifiers used in the composition include cyclopentasiloxane and dimethiconol, and together they are present in a concentration ranging from about 1.0% to 6.0%, preferably about 3.5% by mass based on the total mass of the formulation.

An emulsifier and consistency agent system is preferably a mixture of hydroxyethyl acrylate/sodium acryloyldimethyl taurate copolymer, squalene and polysorbate 60, wherein said mixture is present in a concentration ranging from about 0.1% to 5.0%, preferably about 2% by mass based on the total mass of the formulation.

A pH-correcting agent used is preferably sodium hydroxide or triethanolamine, in a concentration of about 1.85% by mass based on the total mass of the formulation. Said pH-correcting agent is used in a sufficient amount for reaching a pH value of about 7.0.

In order to better perform their function as skin oleosity, said formulations are preferably in the pharmaceutical formulations of aqueous gels, or further as oil-in-water emulsions (o/w). Other agents promoting matter effect, such as Nylon 12, modified starch, tapioca, silica, etc., can also be added.

It must further be emphasized that emulsions and gels of the invention comprise alcohol-free adjuvants or carriers.

Therefore, the present invention provides for a cosmetic formulation which allows for sun protection and comprises anti-ageing sing actives and actives for the treatment of oily, mixed or acned skin, in addition to exhibiting a sensorial feel suitable for this skin type. This fact is still unprecedented in cosmetic market. There have not yet been heard of a product suitable for all these types of skin and providing at the same time all these functions.

Further it is an object of the invention to provide for a kit comprising a composition as described in details above, indicated for diurnal use, and a complementary cosmetic composition, preferably, in the form of an emulsion; and more preferably in the form of serum, indicated for nighttime use, having ultra-light texture, rapid absorption and a final dry and smooth feel, suitable for oily skin. It can further comprises a suitable and balanced combination of actives, promoting immediate and continuous enhancement of an oily skin and providing for the treatment of ageing signs, wherein said actives are for example:
at least a matting agent that absorbs excess of oleosity, thus causing an immediate matte effect;
at least an oleosity control agent for controlling excessive sebum product with continuous use;
at least a pore clearing agent that penetrates the pores to promote a deep exfoliation and aiding their clearing;
at least a hydrating agent that hydrates the skin without leaving it oily; and
at least an anti-ageing sign active promoting an increase in collagen synthesis, regulating elastin production, promoting cellular renovation and redensifying the skin.

With regard to the complementary cosmetic composition, said matting agent is preferably Nylon-12 and is present in a concentration ranging from about 0.1% to 3.0%, preferably about 1.5% by mass based on the total mass of the complementary cosmetic composition.

The preferred oleosity control agent is Zinc PCA is in a concentration ranging from about 0.1% to 2.0%, preferably about 0.70% by mass based on the total mass of the complementary cosmetic composition.

The preferred pore clearing agent of the present invention is salicylic acid that is present in a concentration ranging about 0.1% to 2.0%, preferably about 1% by mass based on the total mass of the complementary cosmetic composition.

The preferred hydrating agent used in the complementary cosmetic composition is glycerin, and is present in a concentration of about 2% by mass or in combination with glycereth-26 in a total concentration of about 7% by weight based on the total mass of the complementary cosmetic composition.

The anti-ageing sign active used in the complementary cosmetic composition of the present invention is a mixture of oligo- and polysaccharide rich in fucose and ramnose, herein designated Elastinol +R, and is present in a concentration ranging from about 0.1% to 10%, preferably about 5% by mass based on the total mass of the complementary cosmetic composition.

Also included in said complementary cosmetic composition are preservatives, emulsifiers, sensorial modifiers, film-forming agent and a pH-correcting agent.

Preferred among said preservatives are phenoxyethanol and methylisothiazolinone in a concentration ranging from about 0.1% to 10.0%, preferably about 0.48% by mass based on the total mass of the complementary composition.

As emulsifier it is preferred to use a mixture of hydroxyethyl acrylate/sodium acryloyldimethyl taurate copolymer, squalene, polysorbate 60 and polysorbate 20. They together are present in a concentration ranging from about 0.1% to 5.0%, preferably about 4% by mass based on the total mass of the complementary cosmetic composition.

Preferred among the sensorial modifiers are cyclopentasiloxane and dimethicone crosspolymer, which are present in a concentration ranging from about 0.1% to 30.0%, preferably about 13% by mass based on the total mass of the complementary cosmetic composition.

As a film-forming agent it is preferred to use a xanthan gum in a concentration ranging from about 0.1% to 1.5%, preferably about 0.7% by mass based on the total mass of the complementary cosmetic composition.

Further it must be emphasized that said complementary cosmetic composition may comprise ethylic alcohol in a concentration ranging from about 0.1% to 20.0%, preferably about 5% by mass based on the total mass of the complementary cosmetic composition.

Some positive characteristics of the actives which can be included in said cosmetic composition for diurnal use and in said complementary cosmetic composition for nighttime use will be highlighted herein.

Glycerin confers hydration on the skin surface, without leaving it oily, due to its ability to retain water. Glycerin is known to be a reference raw material as far as skin hydration is concerned and is present in both cosmetic composition for diurnal use and complementary cosmetic composition for nighttime use. Other hydrating agents that can be used are glycereth-26, propylene glycol and/or butylene glycol.

Zinc PCA is a zinc salt of pyrrolidone carboxylic acid that reduces skin oleosity by biological mechanisms. Said dual action complex associates the benefits of zinc and L-PCA (L-Pirrolidone Carboxylic Acid) that is a physiologic component of the organism. Zinc has the ability to reduce sebaceous secretion by inhibiting enzyme 5-α-reductase. Said enzyme acts through conversion of the hormone responsible for skin oleosity production to its more active form. L-PCA is present in a large amount on the skin surface and is part of the skin hydration system. Other actives, which act inhibiting 5-α-reductase as Zinc gluconate and/or plant extracts, may also be used such as, for example, Camellia sinensis extract, among others.

Sunscreens are used to protect the skin from harmful effects of sun on the skin, such as sunburns (UVB) and photoageing (UVA). Protecting the skin against ultraviolet (UV) radiation means to convert its energy to another form of energy and to guarantee that this other form of energy is not harmful to the skin. Each sunscreen absorbs part of UV (UVA or UVB) region; therefore, in order to achieve full protection a combination of these sunscreens must be made. There is a great trend to use more effective sunscreens which may absorb a higher amount of UV radiation, whereby a broader protection (UVA/UVB) is achieved.

Most sunscreens available in the market are oil-soluble, leaving a heavier sensorial feel in a formulation having SPF thus causing an unpleasant feeling to those having oily skin. In the formulation for oily skin of the present invention hydrosoluble sunscreens are used leaving a totally non-oily formulation with a final dry sensorial feel.

A hydrosoluble UVA screen used in the present invention is disodium phenyl dibenzimidazole tetrasulfonate, and UVB screen is a phenylbenzimidazole sulfonic acid. In addition to these two screens, the screen benphenone-4 is also used. With such a combination of two screens, a synergistic effect is obtained, whereby a complete protection in the whole UV range spectrum is obtained.

Salicylic acid is Beta-Hydro-Acid (BHA) showing keratolytic and also antimicrobial properties. It is used for treating oily skin, acned skin and skin sloughing-off condition (BRACKETT, 1997).

It has the advantage of having a great exfoliating power (chemical exfoliation) by removing death cells from skin surface. Particularly, as it has affinity with oily substances, it mixes with sebum and penetrates the pores causing an effective exfoliation in this environment. Hence, pores are cleared allowing for removal of excessive sebum and reducing pore size (DRAELOS, 2005).

The complementary cosmetic composition of the present invention comprises about 1% salicylic acid that penetrates the pores and clears them so that oleosity is reduced and texture and skin appearance are enhanced.

A mixture of oligo- and polysaccharides rich in fucose and ramnose, designated herein as Elastinol +R is in its turn a patented active and at the present time it is one of the main technologies useful for treating ageing signs, which is present in the commercially available products.

Said active acts on the main skin ageing signs. It is a mixture of polysaccharides (sugars) obtained by biotechnological processes from carbohydrate-rich plant raw materials. More particularly, Elastinol +R is a combination of two raw materials: biosaccharide gum-2 and biosaccharide gum-3.

Elastinol +R acts to regulate elastin production, which is a fiber that confers elasticity on our skin, thus resulting in a more elastic skin (FIG. 2) (ROBERT et al., 2004).

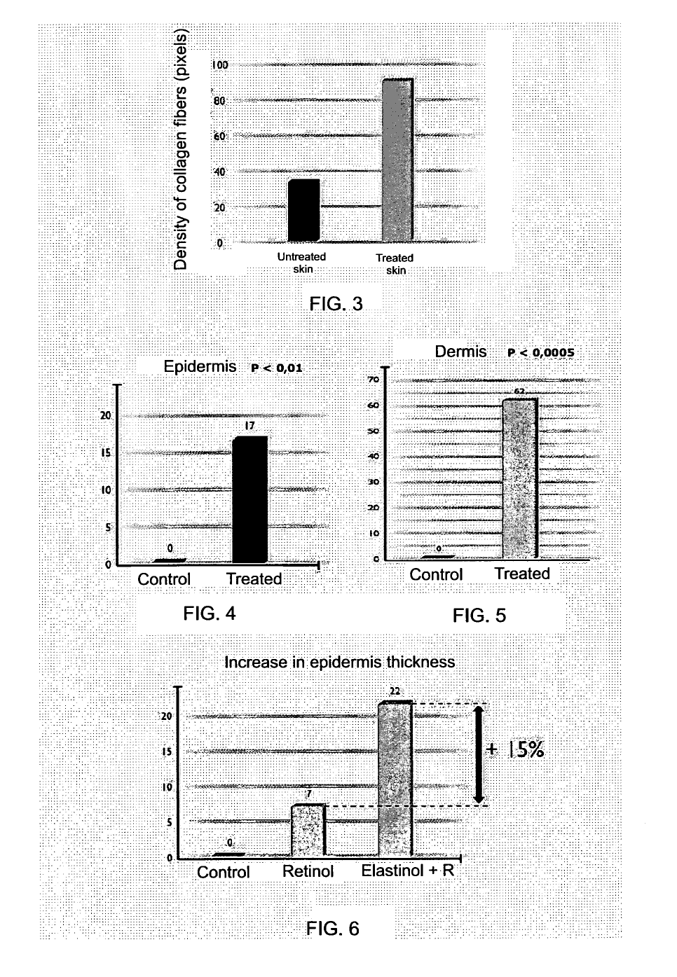

Results of studies carried out on in vivo treatment with emulsion comprising rhamnose-rich sugars and placebo (control) emulsion for five times a week, during four weeks, also demonstrate that Elastinol +R promotes an increase in collagen fiber production on the dermis. This increase in density of dermis collagen fibers enhances the skin firmness (FIG. 3) (FODIL-BOURAHLA et al., 2003).

As the skin ages, it loses the ability to renovate: the number of produced cells is lower, thus causing lost of thickness and lower amount of substances such as collagen, elastin and colloidal gel leading to flaccidity and decrease in skin density (ROBERT et al., 203).

Elastinol +R stimulates renovation of epidermis and dermis (FIGS. 4 and 5). This increase in cellular renovation of the epidermis results in a thickened (FIG. 6), resistant, dense and compacted skin, and restores vitality and smooth appearance to the skin (Report Nos. 2, 4, 7, 8, 20, 22 and 37).

Elastinol +R also acts to stimulate production of important molecules which form an extracellular matrix (also known as colloidal gel), e.g. glucosaminoglycans (ISNARD et al., 2004).

Said glucosaminoglycans are polysaccharide chains formed by two repeated disaccharide units. The structure of these units confer a hydrophilic character on said glycosaminoglycan molecule, thereby aiding in taking up water molecules by said extracellular matrix and providing a higher skin hydration.

By this way, Elastinol +R, due to it broad action on the dermis and epidermis, provides an increase in cutaneous thickness and volume which contribute to filling skin and wrinkles, thus restoring a smooth, soft and healthy appearance to the skin (FODIL-BOURALHA et al., 2003; ISNARD et al., 2004; Report Nos. 2, 4, 7, 8, 20 and 22).

Studies on this active showed that Elastinol +R enhances firmness, elasticity, strength of colloidal gel, cellular redensification, in addition to stimulating cellular renovation of ther dermis.

Therefore, summing up, the benefits of Elastinol +R are improvement in firmness due to collagen production stimulation, in elasticity due to regulation of elastin production, cellular renovation by stimulating epidermis and dermis cellular renovation, and skin redensification by strengthening colloidal gel and increase in skin density.

Finally, another object of the present invention is to provide for an oily, mixed or acned skin treatment method comprising applying said cosmetic composition, preferably in the form of an oil-in-water emulsion (o/w) or an aqueous gel, as defined herein, for diurnal use and subsequently applying said complementary cosmetic composition, preferably in the form of an oil-in-water emulsion (o/w), more preferably in the form of a serum, as defined herein, onto the face skin in the nighttime period.

In order to demonstrate the innocuousness and safety of said cosmetic composition and complementary cosmetic composition for diurnal and nighttime use respectively, tests in humans have been carried out, as represented below:

### a) Evaluation of Cutaneous Compatibility

In accordance with the results obtained form this test, one may conclude that the cosmetic composition and complementary cosmetic composition of the present invention have excellent cutaneous compatibility.

### b) Evaluation of Absence of Allergenic Potential

On the basis of the adopted experimental conditions and results obtained, one may conclude that said cosmetic composition and complementary cosmetic composition of the present invention do not show allergenic potential.

### c) Evaluation of Potential for Cutaneous Photoallergy and Phototoxicity

In accordance with the used methodology and results obtained, one may conclude that the cosmetic composition and complementary cosmetic composition of the present invention are neither photoallergenic nor phototoxic.

### d) Evaluation of Cutaneous Acceptability in Use

In accordance with the adopted experimental conditions and results obtained, one may conclude that the cosmetic composition and complementary cosmetic composition of the present invention show good cutaneous acceptability.

### e) Evaluation of Ocular Acceptability

In accordance with the adopted experimental conditions and results obtained, one may consider that said cosmetic composition and complementary cosmetic composition of the present invention exhibit excellent ocular acceptability.

### f) Evaluation of the Absence of Comedogenic Potential in Use

In accordance with the used methodology and results obtained, one may conclude that said cosmetic composition and complementary cosmetic composition of the present invention can be classified as non-comedogenic.

Hence, it can be concluded that said cosmetic composition and complementary cosmetic composition of the present invention are safe under normal conditions of use.

Cosmetology nowadays may resort to several physical and biological methods for studying the skin, its ageing and also for evaluating effectiveness of the products during skin treatment.

Non-invasive physical methods allow for objectively exploring changes in skin structure and functionality as the skin ages and in the course of treatment, such as, for example, its degree of hydration, among others.

Hydration capacity of the complementary cosmetic composition of the present invention was evaluated in volunteers by using corneometry. This technique determines hydration through objective lenses. A product was applied onto the forearm region of each volunteer, and a control area (without the product) was kept for comparison. Said hydration measurements were performed at zero time and after 2, 12, and 24 hours.

Said complementary cosmetic composition showed an increase in skin hydration (14.8% after 2 hours, 14% after 8 and 12 hours; and 14.7% after 24 hours) and, therefore, the skin was kept hydrated for 24 hours after applying the product.

In order to evaluate reduction in pore size, there has been employed a methodology that determines pore size by means of image analysis. Women having oily skin and visible pores were selected for this study. They used said complementary cosmetic composition during the night and said cosmetic composition during the day, for 30 days, on the facial region. Images by optical microscopy were taken from malar region before applying the product (Tzero) and after 15 min, 1 h, 2 h and 4 h of its application. In the first day (initial), a reduction in pore size by 14.2% 15 minutes after applying the product, 11.5% 1 hour after applying the product, 8.1% 2 hours after applying the product; and 7.0% 4 hours after applying the product occurred.

The results obtained showed that a combined use of formulations significantly reduced the skin pore size after 15 days and 30 days of continuous use of the product compared to the skin pore size prior to this treatment.

Skin oleosity was determined by Sebumetry. Women having oily skin were selected and used on the facial region at night the complementary cosmetic composition and during the day the cosmetic composition, for 30 days. A device capable of detecting the amount of fat on the skin surface (Submeter) was used. Prior to application of the product and 15 minutes, 1 h, 2 h, and 4 h after its application, measurements of oleosity on the volunterrs facial region were measured. This procedure was repeated after 15 and 30 days subsequent to treatment with such products. A significant improvement in the skin oleosity with continuous use of treatment was observed.

In addition to these evaluations effected by means of objective tests, said products may also be subjectively evaluated. Subjective evaluations can be carried out by volunteers by determining the effectiveness of said products under their conditions of use.

Evaluation of perception of effectiveness was performed by volunteers using said cosmetic composition and complementary cosmetic composition of the present invention under real conditions of use. They answered to a standard questionnaire relative to the benefits provided by said product. The volunteers evaluated the product and answered to the questionnaire at Day 0 (initial), Day 7, Day 14, Day 21 and Day 28. All results were assessed by means of statistical methodology and reported in percentage of volunteers who noticed any change in the attributes listed in the questionnaire. Results relative to said cosmetic composition and complementary cosmetic composition of the present invention are shown below on Tables 1 and 2 respectively.

TABLE 1

| Attributes | Day 7 | Day 14 | Day 21 | Day 28 |
|---|---|---|---|---|
| Smoothness | 43 | 71* | 69* | 78* |
| Hydration | 44 | 68 | 58 | 71* |
| Firmness | 46 | 51 | 61 | 69* |
| Elasticity | 32 | 36 | 58 | 59* |
| Density | 27 | 34 | 53* | 61* |
| Oleosity reduction | 81 | 86 | 96 | 100* |
| Glow reduction. | 66 | 73 | 86* | 86* |
| Immediate oleosity | 86 | 88 | 91 | 98* |
| reduction | | | | |
| Dry touch | 41 | 41 | 64* | 75* |
| Pore reduction | 24 | 37 | 46* | 51* |
| Luminosity/Vigor | 34 | 49 | 63* | 64* |
| Uniformity of skin color | 31 | 44 | 61* | 63* |
| Vitality | 46 | 53 | 68 | 66* |
| Texture | 46 | 51 | 65 | 66* |
| Tensor Effect | 68 | 73 | 73 | 73 |
| General Appearance | 71 | 80 | 85* | 90* |

*Value with statistically significant difference

TABLE 2

| Attributes | Day 7 | Day 14 | Day 21 | Day 28 |
|---|---|---|---|---|
| Smoothness | 50 | 77* | 87* | 86* |
| Hydration | 61 | 70* | 89* | 86* |
| Firmness | 46 | 62 | 73* | 71* |
| Elasticity | 35 | 59* | 62* | 60 |
| Oleosity reduction | 89 | 93* | 93* | 96* |
| Glow reduction. | 80 | 89* | 86 | 91 |
| Immediate oleosity | 87 | 94 | 93 | 95* |
| reduction | | | | |
| Dry feel | 39 | 48 | 62* | 62* |
| Luminosity/Vigor | 36 | 50 | 67* | 66* |
| Uniformity of skin color | 57 | 59 | 66* | 75* |
| Texture | 52 | 68 | 78* | 77* |
| Restoration | 50 | 57* | 73 | 77* |
| General Appearance | 77 | 86* | 93* | 94* |

*Value with statistically significant difference

Sun Protection Factor (SPF) of the cosmetic composition was recommended through a methodology recommended by COLIPA. SPF obtained was 16.95 (average of 10 volunteers).

There has also been assessed the behavior of the cosmetic composition of the present invention for sun protection concerning UV-A protection effectiveness by using Standard Australian Method. It was determined that the cosmetic composition presented average transmittance values at 320-360 nm lower than 10% (blockage higher than 90%), whereby the effectiveness of the cosmetic composition of the present invention in terms of its UVA protection was proved.

Finally, photostability of the cosmetic composition of the present invention was tested by a spectrophotometer method. This test was based on measuring UV transmittance radiation through a sample with irradiance fixed on 750 W/m² an periods of time of exposure to UV radiation for 12, 24, 48 and 96 minutes in sun simulator Suntest CPS+(exposure corresponding to 1, 2, 4 and 8 hours of sun radiation required by the present patent application. There have been observed maximal decreases (after 8 hours of sun exposure required by the present patent application) of 0.5% in UVA region and 6.7% in UVB region. It was then concluded that said cosmetic composition of the present application showed high photostability in the UVA and UVB regions.

### BIBLIOGRAPHIC REFERENCES

ABRAMOVITS, W.; GONZALEZ SERVA, A. Sebum, Cosmetics and Skin Care. Dermatol. Clin., v. 18, No. 4, pp. 617-620, 2000.
BRACKETT, W. Therapeutic Use of Salicylic Acid. A Supplement to Cosmetic Dermatology, September, pp. 5-6, 1997.
DRAELOS, Z. D. Cosmeceuticals. 1. Ed. Elsevier Saunders, 2005. p. 228.
FODIL-BOURAHLA, I.; BIZBIZ L.; SCHOEVAERT, B.; ROBERT, A. M.; ROBERT L. Effect of L-fucose and fucose-rich oligo- and polysaccharides (FROP's) on skin aging: penetration, skin tissue production and fibrillogenesis. J Bio Pha, 57: 209-215, 2003.
ISNARD, N.; FODIL-BOURAHLA, I.; ROBERT A M.; ROBERT, L. Pharmacology of skin aging. Stimulation of glycosaminoglycan biosynthesis by L-Fucose and fucose-rich polysaccharides, effect of in vitro aging of fibroblasts. J. Bio. Pha., 58: 202-204, 2004.
NAKAMURA, M. S.; GOMES, J. D.; NUNES, A. S.; GESZTESI, J. Skin Aging Pattern of Brazilian Women. In: 63 rd Annual Meeting American Academy of Dermatology, 2005, New Orleans. Annals, 2005.
PUGLIESE, P. T. Physiology of The Skin Ii: Revised Edition 1. Ed. ALLURED, 2006, p. 383.
REPORT OF RHAMNOSOFT PROJECT No. 2—Effect of in vivo treatment of rat skin on the cellularity of the epidermis. October, 2003.
REPORT OF RHAMNOSOFT PROJECT No. 4—Effect of in vivo treatment of rat skin on the cellularity of the dermis. November, 2003.
REPORT OF RHAMNOSOFT PROJECT No. 7—Effect of Rhamnosoft, of Elastinol and of the mixture of both on the dermis of hairless rat skin. December, 2003.
REPORT OF RHAMNOSOFT PROJECT No. 8—Effect of Rhamnosoft, of Elastinol and of the mixture of both on the thickness of the epidermis of hairless rat skin. December, 2003.
REPORT OF RHAMNOSOFT PROJECT No. 11—Effect of Rhamnosoft on the protein and collagen biosynthesis by human skin fibroblasts. May, 2003.
REPORT OF RHAMNOSOFT PROJECT No. 20—Comparison of the effect of Elastinol, of Rhamnosoft and of the mixture of them on the cellularity of the epidermis. January, 2004.
REPORT OF RHAMNOSOFT PROJECT No. 22—Comparison of the effect of Elastinol, of Rhamnosoft and of the mixture of them on the cellularity of the dermis. February, 2004.
REPORT OF RHAMNOSOFT PROJECT No. 37—Comparison of the effect of Rhamnosoft+Elastinol (50-50%) with retinol on the epidermis of hairless rats by computerized morphometry. June, 2004.
REPORT OF RHAMNOSOFT PROJECT No. 39—Comparison of the effect of Retinol on the epidermis of hairless rats. June, 2004.
ROBERT L.; FODIL-BOURAHLA, I.; BIZBIZ L.; ROBERT, A. M.; Effect of L-fucose and fucose-rich polysaccharides on elastin biosynthesis, in vivo and in vitro. J Bio Pha. 58: 123-128, 2004.
SEGOT-CLICQ, E. et al. Development and validation of a questionnaire to evaluate how a cosmetic product for oily skin is able to improve well-being in women. JEADV, 21, 1181-83, 2007.
ZOUBOULIS C C, BO\ddot{}HM M. Neuroendocrine regulation of sebocytes—a pathogenetic link between stress and acne. Exp. Dermatol., 13 (Suppl. 4): 31-35, 2004.

1. Cosmetic composition comprising comprises:
at least one anti-ageing sign active selected from the group consisting of solubilized hydrophilic and lipophilic actives;
at least a sun protection system selected from the group consisting of hydrophilic sunscreens promoting photostable UVB protection and UVA protection;
at least an oleosity control agent; and
cosmetically acceptable alcohol-free adjuvants or carriers.

2. Cosmetic composition in accordance with claim 1, additionally comprises at least an agent for acne treatment comprising inflammatory and/or anti-septic and/or keratolytic actives.

3. Cosmetic composition in accordance with claim 1 wherein said at least an anti-ageing sign active correspond to about 0.05% to about 10% by mass based on the total mass of the emulsion.

4. Cosmetic composition in accordance with claim 1, wherein said at least a sun protection system provides a protection corresponding to SPF of up to 30.

5. Cosmetic composition in accordance with claim 1, wherein said at least an oleosity control agent comprises Zinc PCA.

6. Cosmetic composition in accordance with claim 1, wherein said composition is for diurnal use.

7. Cosmetic composition in accordance with claim 1 wherein said composition is in the form of an oil-in-water emulsion (o/w).

8. Cosmetic composition in accordance with claim 1, wherein said composition is in the form of an aqueous gel.

9. Skin treatment kit comprising:
(i) a cosmetic composition as defined in claim 1; and
(ii) a complementary cosmetic composition comprising:
at least an anti-ageing sign active selected from the group consisting of solubilized hydrophilic actives and lipophilic actives;
salicylic acid;
at least an oleosity control agent; and
cosmetically acceptable alcohol-containing adjuvants or carriers.

10. Skin treatment kit in accordance with claim 9, wherein the amount of anti-ageing sign active present in the complementary cosmetic composition ranges from about 0.05% to about 10.0% by mass based on total mass of said complementary cosmetic composition.

11. Skin treatment kit in accordance with claim 9, wherein said salicylic is present in an amount of about 0.1% to about 2.0% by mass based on the total mass of the complementary cosmetic composition.

12. Skin treatment kit in accordance with claim 9, wherein said oleosity control agent of the complementary cosmetic composition is Zinc PCA.

13. Skin treatment kit in accordance with claim 9, wherein said complementary cosmetic composition is in the form of serum.

14. Skin treatment kit in accordance with claim 9, wherein said cosmetic composition is indicated for diurnal use and said complementary cosmetic composition is indicated for nighttime use.

15. Cosmetic and/or dermatologic method for the treatment of oily, mixed or acned skin, comprising the steps of:
a) selecting a target area on the skin in which the desired effect is to be obtained;
b) applying onto a previously selected area a cosmetic composition as defined in claim 1 during daylight time.

16. Method in accordance with claim 15, wherein the method comprises the step of:
c) applying onto the previously selected area a complementary cosmetic composition comprising:
at least an anti-ageing sign active selected from the group consisting of solubilized hydrophilic actives and lipophilic actives;
salicylic acid;
at least an oleosity control agent; and
cosmetically acceptable alcohol-containing adjuvants or carriers, at nighttime.
